(19) United States
(12) Patent Application Publication
(10) Pub. No.: US 2024/0299194 A1
(43) Pub. Date: Sep. 12, 2024

(54) MODULAR FOOT CONTROLLED CABLE ACTUATED PROSTHESIS

(71) Applicant: The Chinese University of Hong Kong, Shatin (CN)

(72) Inventors: Darwin Tat Ming LAU, Shatin (CN); Sheung Yan CHAN, Shatin (CN); Elizabeth Ting CHAO, Shatin (CN)

(21) Appl. No.: 18/597,485

(22) Filed: Mar. 6, 2024

Related U.S. Application Data
(60) Provisional application No. 63/450,298, filed on Mar. 6, 2023.

(51) Int. Cl. A61F 2/66; A61F 2/68; A61F 2/70

(52) U.S. Cl. CPC A61F 2/66; A61F 2002/6836; A61F 2002/701

(57) ABSTRACT

Modular systems including a foot controller, cable(s), and prosthetic tool is provided herein. The foot controller can include two sets of wheels that are perpendicularly mounted. Both sets can be coupled to a pulley with a specific gear ratio designed to handle degrees-of-freedom of the tool and having two open loops of cable wrapped around. The cables can be mounted to the pulley and the prosthetic tool on each end. The cables can be Bowden cables that transmit motion and are threaded through a hollow Teflon cable with an outer steel casing. The user can swap cables of different lengths according to the specific task or application of the tool. The prosthetic tool can be mounted to the human body via a socket or brace, which are connected to the cable(s). Either one or both set of cables can be attached to the tool to achieve the intended degrees-of-freedom.

### CROSS-REFERENCES TO RELATED APPLICATION

This application is a Non-Provisional of and claims the benefit of priority to U.S. Provisional Application No. 63/450,298, filed on Mar. 6, 2023, which is incorporated by reference in its entirety.

### FIELD OF INVENTION

The field of invention relates to orthopedic rehabilitation, prosthesis human augmentation and cable actuated prosthesis.

### SUMMARY OF THE INVENTION

In the field of orthopedic rehabilitation, the question is often asked whether it is possible to create a tool that assists in accomplishing tasks that were previously impossible due to one's physical limitations. The invention described herein intends to unlock the possibility of the human body through integrating a tool into the workflow of various tasks. This approach can be particularly useful for people with disabilities, benefitting both able-bodied and disabled persons.

One objective of the invention is a modular system that enables human augmentation. In some embodiments, this is accomplished by a system having a tool or end-effector operatively coupled to a controller. The end-effector can be configured as a prosthesis to perform a task that would typically be performed by a body part replaced by the prosthesis or to augment human function beyond the current or natural function of the body part. The controller can be configured to be operated by a user interaction, for example by physical engagement, such as with a portion of the body, for example an appendage such as a foot, a hand, leg, the user's head, etc. In an exemplary embodiment, the system includes a foot controller coupled to a tool end-effector that functions as prosthesis or human augmentation. A system utilizing a body-part operated controller coupled to a prosthesis end-effector can improve productivity and quality of life.

In some embodiments, this concept can be achieved by coupling omnidirectional wheels, a pulley, and an end-effector together so that users get direct feedback from the end-effector, making it easier and more intuitive to use. In some embodiments, the gear ratio between the end-effector and the omni wheels can be optimized by introducing pulleys with different radii, which enables the possibility of an optimized mechanical advantage for the specific application of various tools.

The invention can provide multiple advantages including: restoring capabilities in users requiring a prosthesis, optimizing and improving the workflow of a specific task, improving quality of life of person with disabilities, and an augmenting human prosthesis. In some embodiments, the system can provide augmentation beyond the ordinary capability of the body part having lost or reduced functionality.

In some embodiments, the modular system includes modular features that allows for assembly of multiple parts, for example, as easy to assemble system with different modular features that allows user to swap in specific parts. In some embodiments, these parts can include any of or any combination of:
1. Controller (e.g. foot controller)—with one or two degrees of freedom with different wheel to pulley gear ratio.
2. Cables—with different lengths according to the desired length and position of the foot controller to the tool end. In some embodiments, Bowden cables can be used.
3. Tool/end-effector—different tools, shapes and mechanisms that are designed to assist or operate for one or more specific tasks that replace or augment human function.
   It is appreciated that these features could be interchanged with various other similar features or mechanisms or could include various other features or mechanisms in addition to those described above.

### BRIEF DESCRIPTION OF DRAWINGS

FIG. 1A show a side view of the system on a user, in accordance with some embodiments.

FIG. 1B show a front view of the system on a user, in accordance with some embodiments.

FIGS. 2A-2C show a top view (FIG. 2a), a side view (FIG. 2b), and a front view (FIG. 2c) view of the foot controller, in accordance with some embodiments.

FIGS. 3A-3C show section views of atop view (FIG. 3a), a side view (FIG. 3b), and a front view (FIG. 3c) of the foot controller, in accordance with some embodiments.

FIGS. 4A-4F show various views of the modular tool changing interface and the adaptor, in accordance with some embodiments.

FIGS. 5A-5C show section views of the modular tool changing interface with the adaptor inserted and FIG. 5D shows an end effector operably coupled to the cables via a modular tool changing interface and adapter, in accordance with some embodiments.

FIGS. 6A-6C each show side and front views of various configuration of the modular tool changing interface, in accordance with some embodiments.

FIGS. 7A-7B illustrate operation of the system along a first degree of freedom, which demonstrates the relationship of motion between the wheel, the cables and the tool, in accordance with some embodiments.

FIGS. 8A-8B illustrate operation of the systems along a second degree of freedom, which demonstrates the relationship of motion between the wheel, the cables and the tool, in accordance with some embodiments.

### DETAILED DESCRIPTION

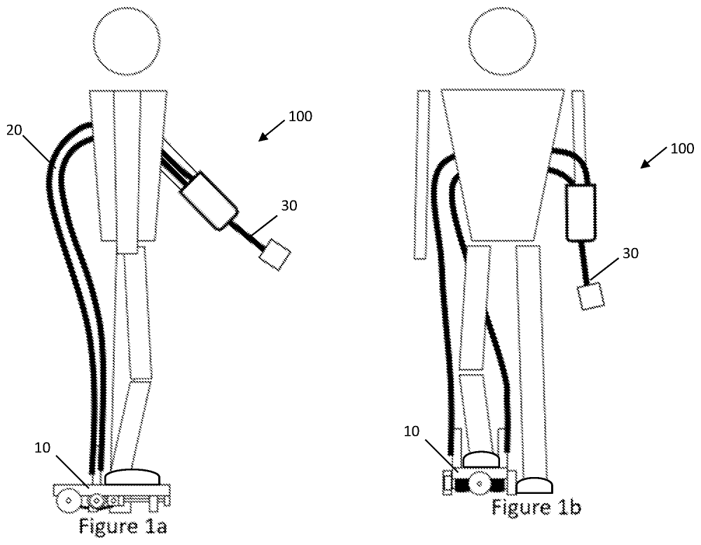

FIGS. 1A-1B show system 100 having a body-part operated controller 10 (e.g. foot controller) that is operatively coupled via cable(s) 20 to a prosthetic tool or end-effector 30. The controller 10 includes wheels by which the user moves the controller and the rotational movement is translated by pulleys 12 into movement of cords or wires in the cables 20 to operate the prosthetic end tool or end effector 30. As shown, the controller 10 is configured for operation by the user's foot. Referring to FIGS. 1A-1B, this system can be assembled according to the specific configuration since one or both pulleys of the foot controller can be utilized by the user.

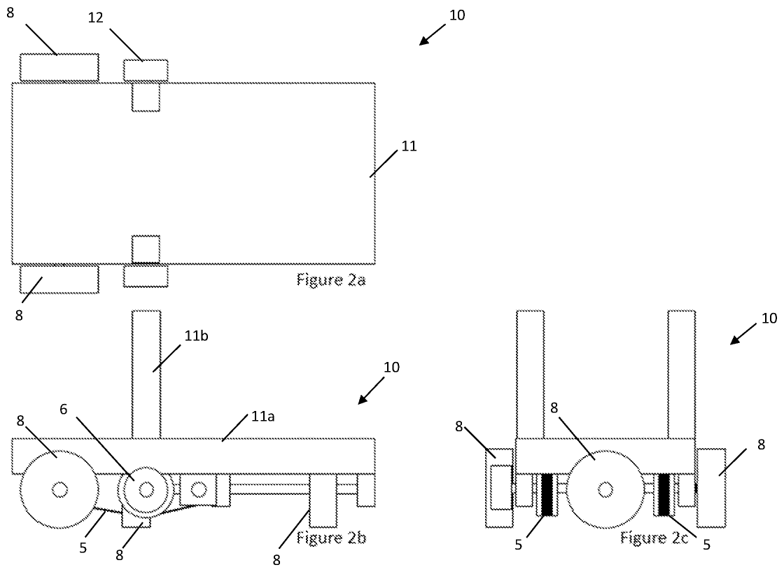

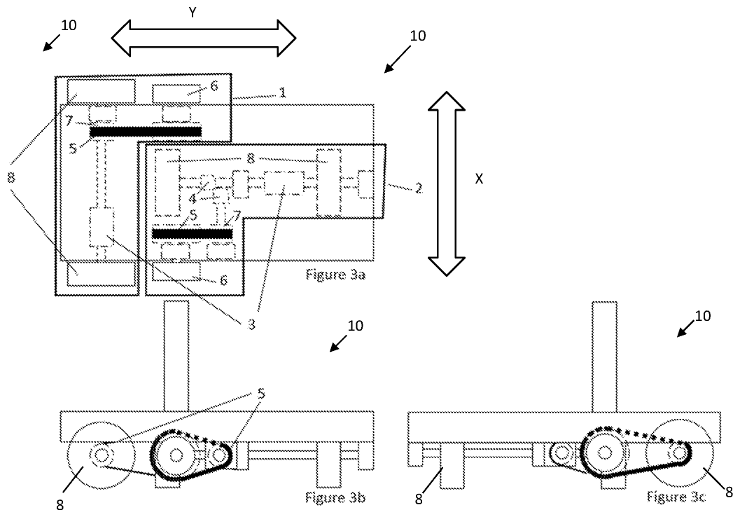

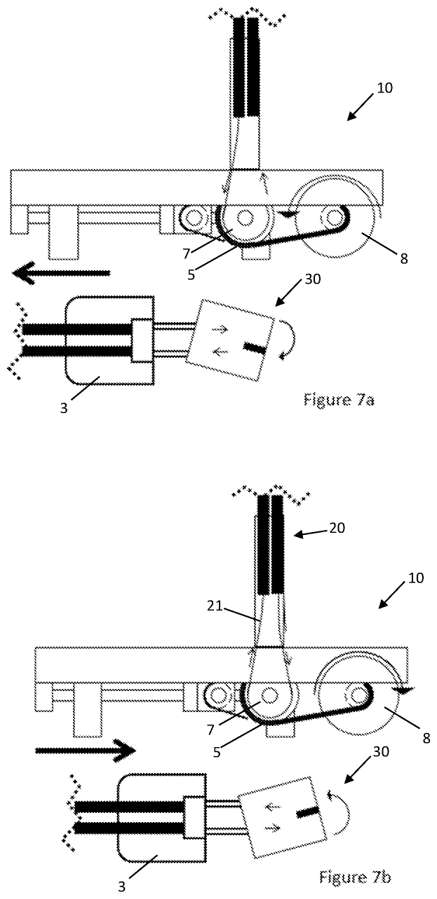

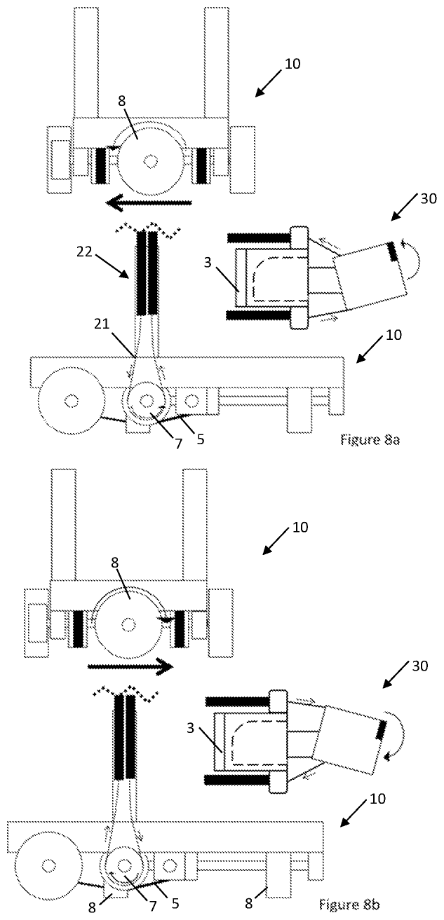

As shown FIGS. 2A-3C, the controller can be configured for use with the user's foot. In this embodiment, the controller includes sets of wheels 8 that, through a system of pulleys 6, gears and timing belt(s) 5, translate the rotation of the wheels 8 to movement of cords or wires extending through cable(s) 20 to operate the end tool or end effector 30. The controller can have a base 11 that is shaped and configured to engage with a body-part to move the controller. In this embodiment, the controller is configured as a foot controller and sized and dimensioned for engagement with the user's foot to transmit forces from push-pull and/or swiping movements to power the tool or end-effector. In this embodiment, the base 11 is configured with a planar base portion 11a and two lateral side portions 11b arranged so that the user's foot fits atop the planar base portion 11 and is partially confined between the two lateral side portions 11b. It is appreciated that the base portion and lateral sides could be configured as an integral part or could include shaped or contoured portions for improved conformance with the body part or for user comfort. This embodiment provides two degrees of freedom, the first degree of freedom provided by the pair of wheels 8 shown in FIG. 2A (push-pull movement along a Y-axis), and the second degree of freedom is provided by a second pair of wheels 8, shown in profile in FIG. 2B (swiping movement along an X-axis). The number of degrees of freedom is determined by the mechanically coupled wheels 8 and pulley 7 through the timing belt 5, as shown in FIG. 3. As the controller is pushed along a surface by the user, the wheels 8 turn, creating tension on the timing belt 5 and turning the pulley 7 on the other end of the belt 5. One or more gears can be used to ratio and/or redirect the movement as needed. The cable includes one or more cords or wires extending therethrough and which can move back and forth. The cords or wires (e.g. Nylon cords) 21 are wrapped around pulley 6 and assert force on the tool through the cable 20 (e.g. Bowden cable), thus actuating the end-effector as shown in FIGS. 7A-7B and 8A-8B. In some embodiments, the workspace ratio between the wheel and the tool can be optimized by switching the pulley 7, as shown in FIG. 3. This changes the gear ratio so that the tool moves intuitively relative to the movement of the foot. In this embodiment, the two degrees of freedom are obtained with either: a) a swiping movement of the foot (e.g. in the X direction), such as shown in FIGS. 8A-8B or; b) a push-pull movement of the foot (e.g. in the Y direction), such as shown in FIGS. 7A-7B. It is appreciated that in other embodiments, the controller could include various other configurations of pulleys and cables and/or various other means by which to adjust the gear ratio or power transmitted by operation of the controller. It is further appreciated that for some end tools or end-effectors only a single degree of freedom may be needed.

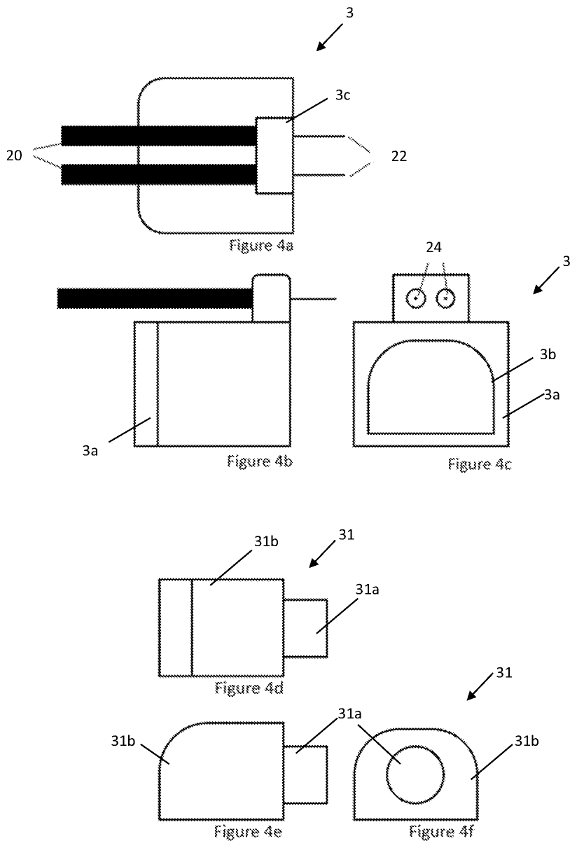

Each degree of freedom is obtained via a separate mechanism as shown in FIG. 3A where Group 1 is responsible for the push-pull movement (along the Y-axis) and Group 2 is responsible for the push-pull movement of the foot (along the X-axis). While the push-pull movement mechanism only has two shafts and two pulleys, the swiping movement mechanism has three shafts, two pulleys and two bevel gears. The shaft that connects the two wheels has a bevel gear 4 that translates the rotational acceleration to the other shaft as shown in FIG. 3A. It is appreciated that various other configuration of wheels, pulleys and gears that perform the same or similar function could be realized. The Bowden cables connect the tool to the foot controller. Nylon wires or cords 21 are fed through the outer casings 21 of the Bowden Cables as shown in FIG. 4A. For a specific application, different lengths of Bowden cables can be coupled to the foot controller to achieve an optimal length and minimum curvature for maximum efficiency of force transmission.

In some embodiments, motors are added to the system for improving the experience during operating. Motors can be added to either to (1) the controller, (2) the end tool, (3) or both according to the application.

In such embodiments, encoders and motors can be mounted and coupled to the shafts for providing haptic feedback and assisting the user for the position control. In some embodiments, the rotational position of the wheels is detected by a microcontroller through the sensors on the motors.

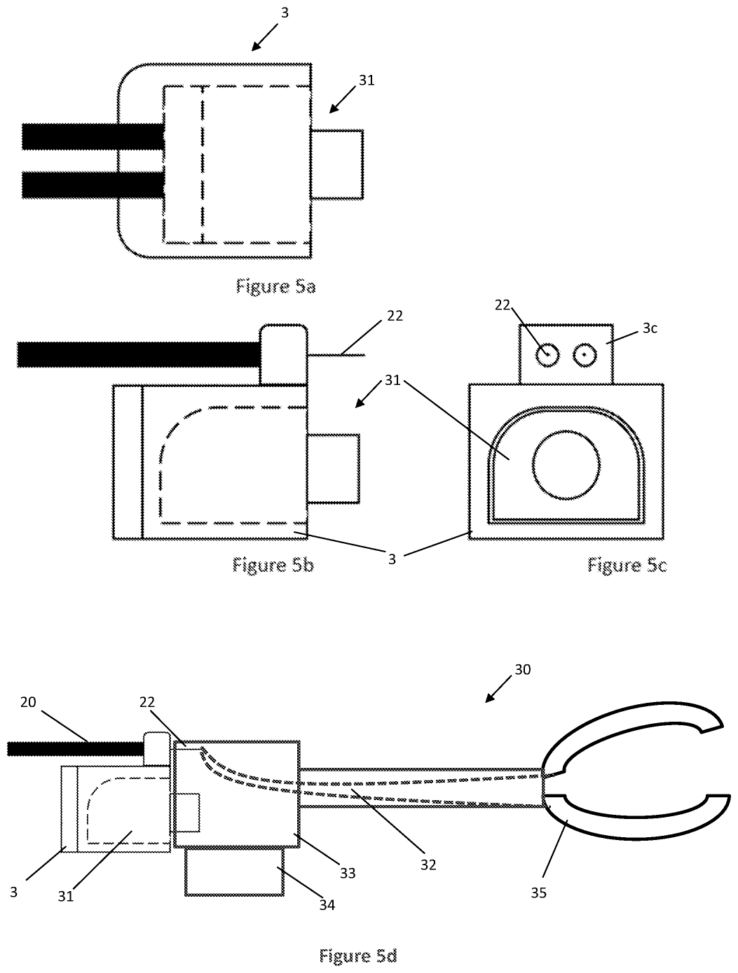

Various different types of tools or end-effectors can be used. In some embodiments, the controller can be used to operate various cable drive tools, either with or without the additional assistance of sets of motors on both the controller and the tool end. In some embodiments, the tool or end-effectors are interchangeable so that the user can select between differing types or sizes of tools or end-effectors. To interchange different tools, the system can include an adapter which allows the connection of various tools to the cables. A connector between the cable of the foot controller and tool allows for modularly connecting the two components. The adapter is designed appropriately to insert the tool, as shown in FIGS. 4A-4F, which show various views of exemplary adapter 3 for interfacing with a corresponding tool adapter 31 of the tool or end-effector 31. In this embodiment, adapter 3 includes a body with a receptacle 3b, and the tool adapter is defined as a plug-like component that is fittingly received within the receptable 3b defined adapter 3. This ensures the tool adapter is properly secured and in a proper orientation so that distal ends 22 of the cords or wires 21 interface with the tool or end effector. Once the tool adapter 31 is plugged into adapter 30, as shown in FIGS. 5A-5D, the distal ends 22 of nylon cables 21 are connected to a separate set of cables that operate the tool or end-effector 30. As shown in FIG. 5D, the tool or end-effector 30 includes a base 33 that includes the tool adapter portion 31 and which includes the proximal ends of the separate set of cables 32 in an orientation and position to releasably couple with the distal ends 22 of the cords or wires of the cable 20 upon being connected with the modular interface adapter. This releasable coupling can be achieved by a friction fit between wires (e.g. distal ends 22 fitting into a receiving sleeve at the proximal ends of the tool wires, interlocking components) or any suitable means. The tool or end-effector can further include a shaft 32 that extends to the distal end effector 35 that is operated by movement of the separate set of cables 32 extending through the shaft. In this embodiment, the distal end effector is depicted as a clamp with jaws that open and close, however, it is appreciated that various differing types and sizes of tools or end-effectors (e.g. clamps, graspers, prosthetic hands, etc.) can be used and operated by the same concepts. In some embodiments, the tool adapter can be incorporated into the base of the tool, and in other embodiments, the tool adapter can be separable from the tool. In some embodiments, the tool can include a user body coupler 34 for releasably securing the tool or end-effector to the body of the user, for example, an arm cuff or sleeve to secure the end-effector to the forearm of the user so that the clamp can replace or augment the function of the user's hand. As the system is mechanically coupled, the user can sense feedback from the end-effector via the foot controller.

For one version of the design, the tool is passively controlled by the foot controller, and the amount of movement in either one or two degrees of freedom is controlled directly by the cables and pulleys attached to the system.

Additional motors can be added to the tool end for any or all of the purposes of: (1) guiding the tool's movement or trajectory, (2) stabilization of the tool's movement, (3) increasing the range of the workspace of the tool since the foot's workspace is limited, and (4) allowing for human interaction and human intention control.

Furthermore, this foot controller can be used for mechanically coupled tools and teleoperated tools. The foot controller can output position data which can be used for the input of tools.

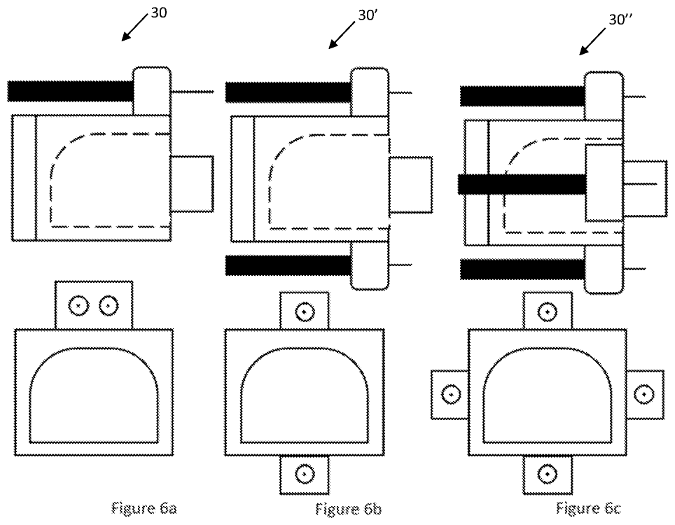

FIGS. 6A-6C show additional variations of the adapter of the modular tool changing interface, that allow for interfacing of additional sets of cables between the controller and the tool or end-effector as needed for differing types of tools or end-effectors. FIG. 6A shows the exemplary adapter 30 that supports one set of cables having two wires or cords mounted on one side of the adapter. FIG. 6B shows another adapter 30′ that supports one set of cables having two wires or cords that are disposed on opposite sides of the adapter. FIG. 6C shows an adapter 3″ that supports two sets of cables, each having two wires or cords, each wire being mounted on a different side of the adapter. The adapters 30, 30′ supporting one set of cables support one degree of freedom. The adapter 30″ interfacing with two sets of cables supports two degrees of freedom. These adapters allow for use of differing tools or end-effector, which can include additional degrees of freedom, multiple tools, or variable force capabilities. Additionally, these adapter could be used to couple with one or more controllers and/or one or more tools or end-effectors, each optionally being engage with different body parts of the user. It is appreciated that still further variations of the adapter could be realized.

FIGS. 7A-7B and 8A-8B demonstrate the mechanisms of action by which user operation of the controller effect actuations of the tool or end effector. These figures demonstrate user operation of the foot controller 10 described above for actuation of tool or end effector 30 having two degrees of movement.

FIGS. 7A-7B demonstrate how a push-pull movement of the foot controller 10 (e.g. in the Y direction) effects actuation of the tool or end-effector 30 operably coupled to the controller by a set of cables 20. Upon a pull movement of the foot controller 10 on a surface, as shown at top in FIG. 7A, wheels 8 are rotated in one direction, thereby moving belt 5, which via one or more gears rotates pulley 7, thereby moving cords 21 in the set of cables 20. This movement of the cords 21 is transmitted to the other end of the set of cables 20 and effects movement of the distal end effector 30 (or internal component thereof) in a first direction, as shown at bottom in FIG. 7A. Upon a push movement of the foot controller 10 on a surface, as shown at top in FIG. 7B, wheels 8 rotate in the opposite direction, thereby moving belt 5, which via one or more gears rotates pulley 7 in the opposite direction, thereby moving cords 21 in the set of cables 20 in the opposite direction. This movement of the cords 21 is transmitted through the set of cables 20 and effects movement of the distal end effector 30 (or internal component thereof) in the opposite direction as the first direction along the same first degree of freedom.

FIGS. 8A-8B demonstrate how a swiping movement of the foot controller 10 (e.g. in the X direction) effects actuation of the tool or end-effector 30 operably coupled to the controller by the set of cables 20. Upon a swiping movement of the foot controller 10 on a surface in the left lateral direction, as shown at left in FIG. 8A, wheels 8 are rotated, thereby moving belt 5, which via one or more gears rotates pulley 7, thereby moving cords 21 in the set of cables 20. This movement of the cords 21 is transmitted to the other end of the set of cables 20 and effects movement of the distal end effector 30 (or internal component thereof) in a second direction (along a second degree of freedom), as shown at right in FIG. 8A. Upon a swiping movement of the foot controller 10 on a surface in a right lateral direction, as shown at left in FIG. 8B, wheels 8 rotate in the opposite direction, thereby moving belt 5, which via one or more gears rotate pulley 7 in the opposite direction, thereby moving the cords 21 in the set of cables 20 in the opposite direction. This movement of the cords 21 is transmitted through the set of cables 20 to the opposite end and effects movement of the distal end effector 30 (or internal component thereof) in the opposite direction along the same second degree of freedom.

In the foregoing specification, the invention is described with reference to specific embodiments thereof, but those skilled in the art will recognize that the invention is not limited thereto. Various features, embodiments and aspects of the invention can be used individually or jointly and can be utilized in any number of environments and applications beyond those described without departing from the broader spirit and scope of the specification. The specification and drawings are to be regarded as illustrative rather than restrictive. The terms “comprising,” “including,” and “having,” are specifically intended to be read as open-ended. Any references to publications or patent applications are incorporated herein for all purposes.

What is claimed:

1. A system for operating an actuated end-effector, the system comprising:
a controller configured to be operated by a body part of the user;
one or more cables; and
a cable-driven end tool operably coupled with the controller by the one or more cables such that operation by the controller by the user effects operation of the tool via the one or more cables.

2. The system of claim 1, wherein the end tool is an end effector.

3. The system of claim 1, wherein the one or more cables comprise Bowden cables.

4. The system of claim 1, further comprising one or more motors.

5. The system of claim 4, wherein the controller is configured to be operated by the user's foot.

6. The system of claim 4, wherein the tool is configured to be mounted on a user's arm for use as a prosthetic or human augmentation.

7. The system of claim 5, wherein the one or more motors are mounted and coupled to both the foot controller and the end tool and/or the foot controller.

8. The system of claim 5, wherein the one or more motors of the foot controller are configured to assist and generate haptic feedback to user.

9. The system of 5, wherein the foot controller comprises wheels that enable movement.

10. The system of claim 9 wherein the wheels comprise sets of wheels that are independent from each other's movement and configured for the actuation of the end tool.

11. The system of claim 10 wherein the wheels comprise a first set of wheels configured to move in a first direction and a second set of wheels configured to move in a second direction different than the first direction.

12. The system of claim 13 wherein the first and second directions are orthogonal.

13. The system of claim 9 further comprising one or more pulleys mechanically coupled to the wheels and the motor.

14. The system of claim 13 wherein the cables comprise Bowden cables and each of the Bowden cables comprises an outer housing and an inner cord or wire.

15. The system of claim 14, wherein the Bowden cables can be coupled with either or both the pulley of the foot controller and the end tool.

16. The system of claim 14, wherein the Bowden cables transmit mechanical force from the foot controller to the end tool.

17. The system of claim 14, wherein the end tool is coupled with the Bowden cables.

18. The system of claim 1 wherein the one or more cables comprises a plurality of cables and the end tool comprises an end-effector.

19. The system of claim 1 wherein the end-effector is coupled to multiple sets of cables configured to actuate the end-effector along multiple degrees of freedom.

20. The system of claim 19 wherein the end-effector comprises one or more joints configured to allow the movement along the multiple degrees of freedom.
